(19) United States
(12) Patent Application Publication
(10) Pub. No.: US 2009/0187071 A1
(43) Pub. Date: Jul. 23, 2009

(54) CAPSULE ENDOSCOPE SYSTEM AND METHOD FOR IMPLEMENTING TIME SHIFT FUNCTION THEREIN

(75) Inventor: Iy Kon KIM, Seoul (KR)

Correspondence Address: Workman Nydegger;1000 Eagle Gate Tower, 60 East South Temple, Salt Lake City, UT, 84111, US

(73) Assignee: INTROMEDIC CO., LTD., Seoul (KR)

(21) Appl. No.: 12/058,688

(22) Filed: Mar. 29, 2008

(30) Foreign Application Priority Data
Jan. 23, 2008 (KR) 10-2008-0006999

(51) Int. Cl. A61B 1/045; A61B 1/04

(52) U.S. Cl. 600/109; 600/118

(57) ABSTRACT

A capsule endoscope system and related method for capturing an image of the inside of a human body using a capsule endoscope and storing or displaying the captured image in real time to implement a time shift function. The endoscopy capsule captures an image of an inside of a human body. The captured image is transmitted to an image processing unit and is displayed in real time. The image is stored in a storage unit within a predetermined storage range for the time shift function. When the time shift function is requested by a user, a progress bar is displayed, showing a progress state of the storage of the transmitted image in real time by means of an indicator. When the user moves the indicator to a past time using a user interface, a time-shifted image is displayed on the display beginning with the past time.

This application claims the benefit of the Korean Patent Application No. 10-2008-06999, filed on Jan. 23, 2008, which is hereby incorporated by reference as if fully set forth herein.

### BACKGROUND OF THE INVENTION

1. Field of the Invention

The present invention relates to a capsule endoscope system, and more particularly, to a capsule endoscope system for capturing an image of the inside of a human body using a capsule endoscope and storing or displaying the captured image in real time to implement a time shift function, and a method for implementing the time shift function therein.

2. Discussion of the Related Art

Recently, a capsule-type endoscope has been developed and used to diagnose various diseases in medical fields.

The capsule-type endoscope is a tablet-sized endoscope that can be swallowed by a person, and then capture an image of the inside of the body of the person and transmit the captured image to an external device through wireless communication. This capsule-type endoscope is advantageous in that anesthesia is not required, there is no nausea and a precise diagnosis can be made of even the small intestines inaccessible by existing endoscopes.

Such a capsule endoscope captures an image of the inside of a human body through an image sensor and displays the captured image on a screen in real time so that an observer can observe abnormalities inside the human body.

However, when the observer wants to view a past scene again while displaying the real-time image and observing abnormalities inside the human body, as stated above, he/she cannot immediately play back and view the past scene.

Also, the observer can record a desired portion of the real-time image using a separate recording device, but cannot play back the desired portion during the recording. That is, the playback can be performed after the entire recording is completed.

Further, when the observer wants to again view a past scene of a stored image, he/she has difficulty in searching for a position of the past scene.

In addition, in a recording function, it is impossible to simultaneously display a recorded image and a current image, received in real time, on one screen. Moreover, it is impossible to shift the display from a recorded image to a currently received image at a time desired by the observer during playback of the recorded image, or shift the display from a currently received image to a recorded image during the display of the currently received image.

Furthermore, it is impossible to provide various information related to an image displayed on a screen together with the image.

### SUMMARY OF THE INVENTION

Accordingly, the present invention is directed to a capsule endoscope system and a method for implementing time shift function therein that substantially obviate one or more problems due to limitations and disadvantages of the related art.

An object of the present invention is to provide a capsule endoscope system for capturing an image of the inside of a human body using a capsule endoscope and storing or displaying the captured image in real time to implement a time shift function, and a method for implementing the time shift function therein.

Another object of the present invention is to provide a capsule endoscope system for, when an observer wants to again view a past scene of a stored image, displaying an indicator to enable the observer to conveniently search for a playback position of the past scene.

A further object of the present invention is to provide a capsule endoscope system for displaying a current real-time image and a time-shifted image on one screen in various ways, and displaying representative images and details of a specific image on one screen to provide detailed information regarding the specific image.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, in a display method of a capsule endoscope system including a capsule endoscope and an image processing unit, a method for implementing a time shift function in the capsule endoscope system comprises: the endoscopy capsule capturing an image of an inside of a human body and transmitting the captured image to the image processing unit, the image processing unit being located outside the human body; the image processing unit processing the transmitted image to display it on a display in real time; storing the transmitted image in a storage unit of the image processing unit within a predetermined storage range for the time shift function; when the time shift function is requested by a user, displaying, on the display, a progress bar showing a progress state of the storage of the transmitted image within the predetermined storage range in real time by means of an indicator; and when the user moves the indicator to a past time using a user interface, displaying a time-shifted image on at least part of the display beginning with the past time.

The display may comprise a single screen. In this case, the at least part of the display may be half an entire area of the screen. Alternatively, the at least part of the display may be smaller than half an entire area of the screen.

The method for implementing the time shift function in the capsule endoscope system may further comprise, in response to a request from the user using the user interface, displaying the time-shifted image on an entire area of the screen without displaying the real-time image.

As an alternative, the display may comprise two screens, one used to display the real-time image and the other used to display the time-shifted image.

The storage unit may comprise a hard disk driver, and the predetermined storage range may be preset to a certain storage area of the hard disk driver.

The method for implementing the time shift function in the capsule endoscope system may further comprise, in response to a request from the user, storing all images captured by the endoscopy capsule in a separate area of the storage unit different from a storage area for the time shift function.

In another aspect of the present invention, a capsule endoscope system comprises: a swallowable endoscopy capsule for capturing an image of an inside of a human body and transmitting the captured image externally; an image processing unit including a controller for generating a plurality of control signals, a storage unit for permanently storing the transmitted image and/or temporarily storing it for a time shift function, under control of the controller, and a signal processor for processing the transmitted image so that it can be displayed, under the control of the controller; and a display for displaying at least one of the permanently stored image and temporarily stored image under the control of the controller.

The storage unit may comprise: a hard disk driver for permanently storing the transmitted image and/or temporarily storing it for the time shift function; and a personal video recorder (PVR) controller for controlling the hard disk driver under the control of the controller.

The capsule endoscope system may further comprise an on-screen display (OSD) generator for generating, for the time shift function, a progress bar showing a progress state of the storage of the transmitted image in the storage unit within a predetermined storage range.

The progress bar may comprise: a cache bar having a length corresponding to the predetermined storage range, the cache bar showing the progress state; and an indicator for indicating a current storage location on the cache bar.

The progress state may be displayed on the cache bar using at least one of shading and coloring.

The display may comprise: one or more representative image windows, each of the one or more representative image windows displaying a corresponding one of one or more representative images of a specific image; and one or more information windows, each of the one or more information windows displaying a title, time and related information of the representative image displayed in a corresponding one of the one or more representative image windows.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a block diagram showing the configuration of a capsule endoscope system according to one embodiment of the present invention;

FIG. 2 is a detailed block diagram of an image processing unit in the capsule endoscope system of FIG. 1;

FIG. 3 is a detailed block diagram of a capsule endoscope in the capsule endoscope system of FIG. 1;

FIG. 4 is a flowchart illustrating a method for implementing a time shift function in a capsule endoscope system, according to one embodiment of the present invention;

FIGS. 5 to 8 are screen configuration diagrams illustrating various processes of displaying a live image and a time-shifted image, according to one embodiment of the present invention; and

FIG. 9 is a screen configuration diagram illustrating a process of displaying representative images and details of a specific image, according to one embodiment of the present invention.

### DESCRIPTION OF SPECIFIC EMBODIMENTS

Reference will now be made in detail to the preferred embodiments of the present invention, examples of which are illustrated in the accompanying drawings. Wherever possible, the same reference numbers will be used throughout the drawings to refer to the same or like parts. In the following description of the present invention, a detailed description of known functions and configurations incorporated herein will be omitted when it may make the subject matter of the invention rather unclear.

Besides, although terms used in the present invention are possibly selected from the currently well-known ones, some terms are arbitrarily chosen by the inventor in some cases so that their meanings are explained in detail in the following description. Hence, the present invention should be understood with the intended meanings of the corresponding terms chosen by the inventor instead of the simple names or meanings of the terms themselves.

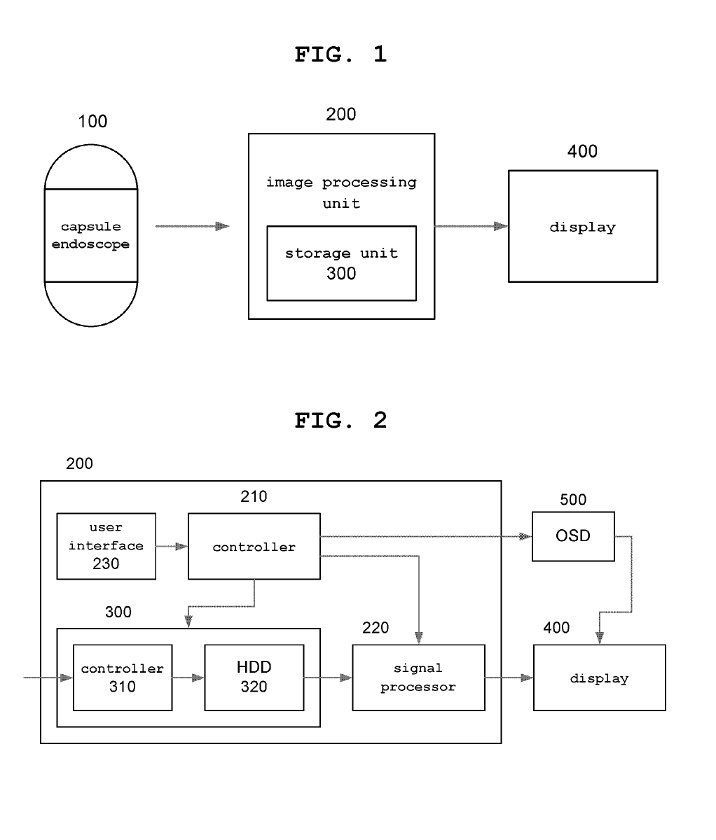

FIG. 1 is a block diagram showing the configuration of a capsule endoscope system according to one embodiment of the present invention.

As shown in FIG. 1, the capsule endoscope system according to the present embodiment comprises a capsule endoscope 100, image processing unit 200, storage unit 300, and display 400.

The endoscopy capsule 100 is swallowable, and captures an image of the inside of a human body and transmits the captured image externally.

The image processing unit 200 controls the storage unit 300 and display 400, and processes the captured image transmitted from the endoscopy capsule 100 so that it can be displayed.

The storage unit 300 permanently stores the transmitted image and/or temporarily stores it for a time shift function, under the control of the image processing unit 200.

The storage unit 300 is preferably a personal video recorder (PVR).

The display 400 displays at least one of the permanently stored image and temporarily stored image under the control of the image processing unit 200.

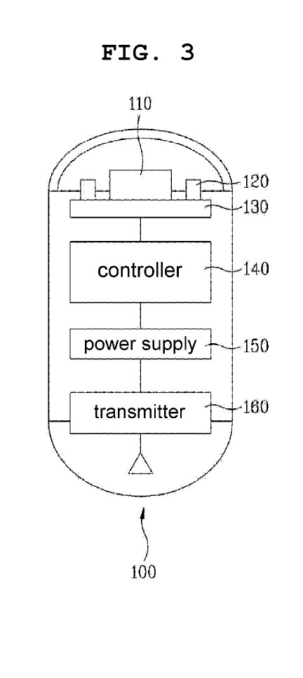

FIG. 2 is a detailed block diagram of the image processing unit 200.

As shown in FIG. 2, the image processing unit 200 includes a controller 210, signal processor 220, and user interface 230.

The user inputs a control command to the controller 210 through the user interface 230, and the controller 210 generates a control signal in response to the inputted control command. The signal processor 220 processes the captured image transmitted from the endoscopy capsule 100 so that it can be displayed, under the control of the controller 210.

The controller 210 is a typical processor having computation, comparison and determination functions, such as a microcomputer, microprocessor, central processing unit (CPU) or microprocessor unit (MPU).

The storage unit (PVR) 300 includes a PVR controller 310 and a hard disk driver (HDD) 320. The hard disk driver 320 includes a magnetic recording system and an optical recording system.

The PVR controller 310 controls the hard disk driver 320 under the control of the controller 210.

The hard disk driver 320 permanently stores the transmitted image and/or temporarily stores it for the time shift function, under the control of the PVR controller 310.

As a result, the display 400 displays at least one of the permanently stored image and temporarily stored image under the control of the controller 210.

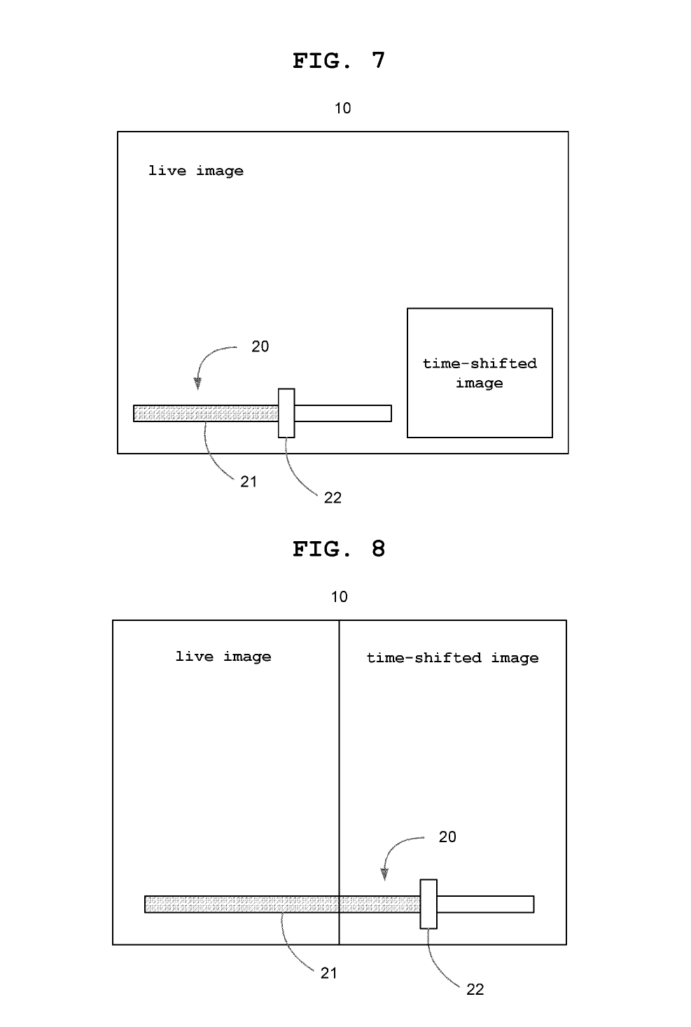

The capsule endoscope system according to the present embodiment further comprises an on-screen display (OSD) generator 500. The OSD generator 500 generates, for the time shift function, a progress bar 20 (see FIG. 5) showing a progress state of the storage of the captured image transmitted from the endoscopy capsule 100 in the PVR 300 within a predetermined storage range.

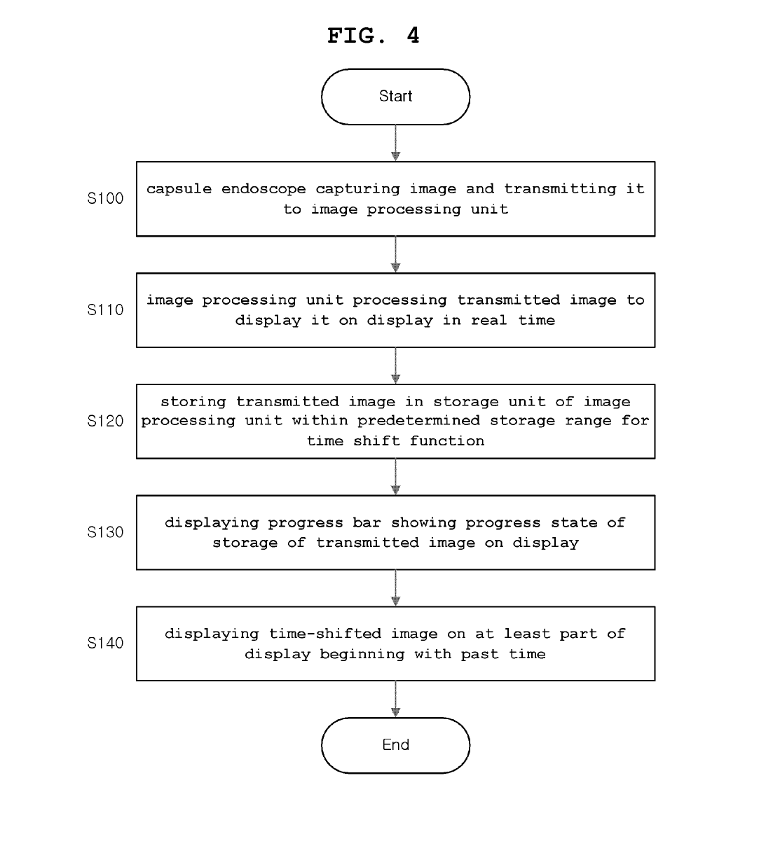

FIG. 3 is a detailed block diagram of the endoscopy capsule 100.

As shown in FIG. 3, the endoscopy capsule 100 includes a lens 110, light source 120, image sensor 130, controller 140, power supply 150, and transmitter 160.

The light source 120 emits light externally, and the lens 110 condenses the light from the light source 120. The light source 120 is tuned on/off in response to a control signal from the controller 140, and may be, for example, a light emitting diode (LED).

The image sensor 130 converts light incident through the lens 110 into an image. The image sensor 130 captures an image based on an on/off state of the light source 120. That is, the image sensor 130 can capture a light image in the on state of the light source 120 and a no-light image in the off state of the light source 120. The image sensor 130 may be implemented with, for example, a charge coupled device (CCD) sensor or complementary metal oxide semiconductor (CMOS) sensor.

The controller 140 controls the turning-on/off of the light source 120, and transmits an image received from the image sensor 130 to the external processing unit 200 through the transmitter 160.

The power supply 150 is a constituent element that supplies power to the endoscopy capsule 100.

The transmitter 160 is a communication unit that transmits an image to the image processing unit 200 under the control of the controller 140. The transmitter 160 may employ a general radio communication system.

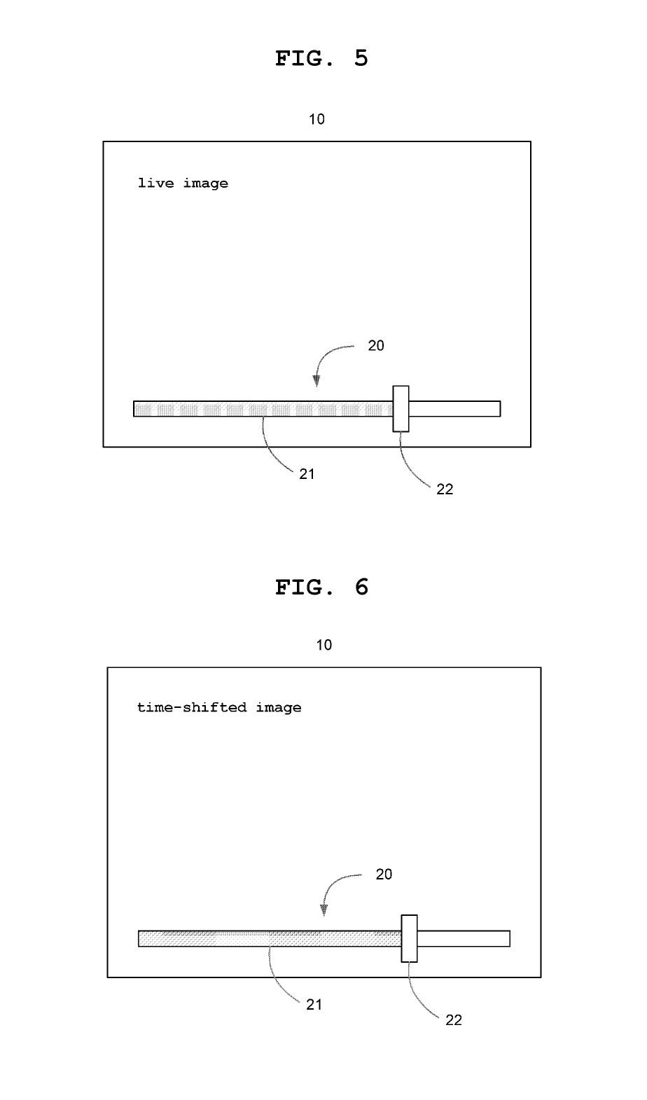

FIG. 4 is a flowchart illustrating a method for implementing a time shift function in a capsule endoscope system, according to one embodiment of the present invention.

As shown in FIG. 4, in a display method of the capsule endoscope system including the endoscopy capsule 100 and image processing unit 200, in the time shift function implementation method of the capsule endoscope system according to the present embodiment, the endoscopy capsule 100 captures an image of the inside of a human body and transmits the captured image to the image processing unit 200, located outside the human body (Step S100).

At this time, the image capture function of the endoscopy capsule 100 is performed in the following manner. That is, as shown in FIG. 3, the lens 110 of the endoscopy capsule 100 condenses light from the light source 120, the image sensor 130 converts light incident through the lens 110 into an image, and the controller 140 controls turning-on/off of the light source 120, and transmits an image received from the image sensor 130 to the external image processing unit 200 through the transmitter 160.

Thereafter, the image processing unit 200 processes the transmitted image to display it on the display 400 in real time (Step S110).

That is, the controller 210 of the image processing unit 200 generates a plurality of control signals, and the signal processor 220 processes the transmitted image so that it can be displayed, under the control of the controller 210. The display 400 displays the processed image under the control of the controller 210.

Thereafter, for the time shift function, the transmitted image is stored in the storage unit 300 of the image processing unit 200 within a predetermined storage range (Step S120).

As stated previously, the storage unit (PVR) 300 includes the PVR controller 310 and the hard disk driver (HDD) 320.

The PVR controller 310 controls the hard disk driver 320 under the control of the controller 210, and the hard disk driver 320 permanently stores the transmitted image and/or temporarily stores it for the time shift function, under the control of the PVR controller 310.

When the time shift function is requested by the user, a progress bar 20 showing a progress state of the storage of the transmitted image within the predetermined storage range in real time by means of an indicator 22 is displayed on the display 400 (Step S130).

At this time, the OSD generator 500 generates, for the time shift function, the progress bar 20 showing the progress state of the storage of the transmitted image in the PVR 300 within the predetermined storage range.

Thereafter, when the user moves the indicator 22 to a past time using the user interface 230, a time-shifted image is displayed on at least part of the display 400 beginning with the past time (Step S140).

Preferably, the display 400 includes a single screen 10, and the at least part of the display is half the entire area of the screen 10 (see FIG. 7) or is smaller than half the entire area of the screen 10 (see FIG. 6).

FIGS. 5 to 8 are screen configuration diagrams illustrating various processes of displaying a live (real-time) image and a time-shifted image, according to one embodiment of the present invention.

As shown in these drawings, a progress bar 20, cache bar 21, and indicator 22 are displayed on a screen 10.

The progress bar 20 includes the cache bar 21 and the indicator 22. The cache bar 21 has a length corresponding to the predetermined storage range, and shows the progress state of image storage. The indicator 22 indicates a current storage location on the cache bar 21.

The progress state is displayed on the cache bar 21 using at least one of shading and coloring.

On the other hand, the time shift function implementation method of the capsule endoscope system according to the present embodiment may further include a step of, in response to a request from the user using the user interface 230, displaying a time-shifted image on the entire area of the display without displaying a live image, as shown in FIG. 8.

As shown in FIGS. 6 and 7, the display may include two screens, one used to display the live image and the other used to display the time-shifted image.

On the other hand, the storage unit 300 includes the hard disk driver 320 as stated previously, and the predetermined storage range may be preset to a certain storage area of the hard disk driver 320.

The time shift function implementation method of the capsule endoscope system according to the present embodiment may further include a step of, in response to a request from the user, storing all images captured by the endoscopy capsule 100 in a separate area of the storage unit 300 different from the storage area for the time shift function.

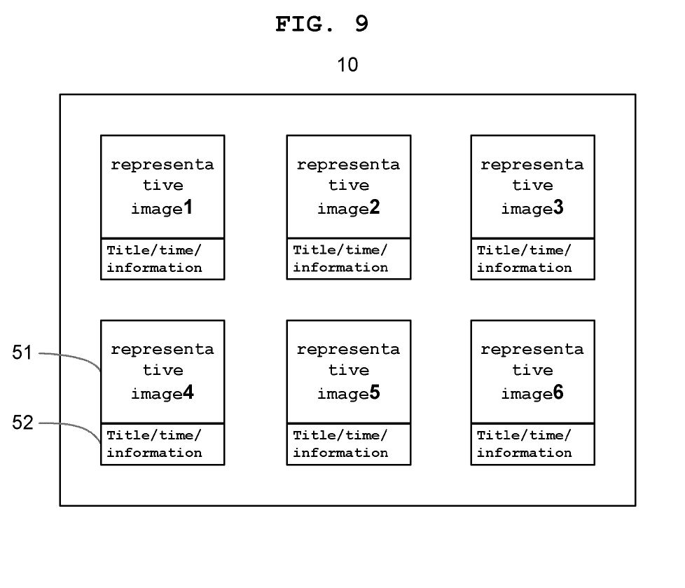

FIG. 9 is a screen configuration diagram illustrating a process of displaying representative images and details of a specific image, according to one embodiment of the present invention.

As shown in FIG. 9, one or more representative image windows 51 and one or more information windows 52 are provided on a screen 10.

Each of the one or more representative image windows 51 displays a corresponding one of one or more representative images of the specific image, and each of the one or more information windows 52 displays a title, time and related information of the representative image displayed in the corresponding representative image window 51.

The one or more representative image windows 51 may be displayed on the screen 10 in the form of blocks arranged in various forms, for example, in tiers or paduk board form.

Accordingly, the user can receive detailed information about the specific image by displaying the representative images and details of the specific image. Also, the user can play back the specific image immediately by selecting any one of the one or more representative image windows 51.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. In a display method of a capsule endoscope system including a capsule endoscope and an image processing unit, a method for implementing a time shift function in the capsule endoscope system, comprising:
the endoscopy capsule capturing an image of an inside of a human body and transmitting the captured image to the image processing unit, the image processing unit being located outside the human body;
the image processing unit processing the transmitted image to display it on a display in real time;
storing the transmitted image in a storage unit of the image processing unit within a predetermined storage range for the time shift function;
when the time shift function is requested by a user, displaying, on the display, a progress bar showing a progress state of the storage of the transmitted image within the predetermined storage range in real time by means of an indicator; and
when the user moves the indicator to a past time using a user interface, displaying a time-shifted image on at least part of the display beginning with the past time.

2. The method for implementing the time shift function in the capsule endoscope system, according to claim 1, wherein the display comprises a single screen.

3. The method for implementing the time shift function in the capsule endoscope system, according to claim 2, wherein the at least part of the display is half an entire area of the screen.

4. The method for implementing the time shift function in the capsule endoscope system, according to claim 2, wherein the at least part of the display is smaller than half an entire area of the screen.

5. The method for implementing the time shift function in the capsule endoscope system, according to claim 2, further comprising, in response to a request from the user using the user interface, displaying the time-shifted image on an entire area of the screen without displaying the real-time image.

6. The method for implementing the time shift function in the capsule endoscope system, according to claim 1, wherein the display comprises two screens, one used to display the real-time image and the other used to display the time-shifted image.

7. The method for implementing the time shift function in the capsule endoscope system, according to claim 1, wherein the storage unit comprises a hard disk driver, and the predetermined storage range is preset to a certain storage area of the hard disk driver.

8. The method for implementing the time shift function in the capsule endoscope system, according to claim 1, further comprising, in response to a request from the user, storing all images captured by the endoscopy capsule in a separate area of the storage unit different from a storage area for the time shift function.

9. A capsule endoscope system comprising:
a swallowable endoscopy capsule for capturing an image of an inside of a human body and transmitting the captured image externally;
an image processing unit including a controller for generating a plurality of control signals, a storage unit for permanently storing the transmitted image and/or temporarily storing it for a time shift function, under control of the controller, and a signal processor for processing the transmitted image so that it can be displayed, under the control of the controller; and
a display for displaying at least one of the permanently stored image and temporarily stored image under the control of the controller.

10. The capsule endoscope system according to claim 9, wherein the storage unit comprises:
a hard disk driver for permanently storing the transmitted image and/or temporarily storing it for the time shift function; and
a personal video recorder (PVR) controller for controlling the hard disk driver under the control of the controller.

11. The capsule endoscope system according to claim 9, further comprising an on-screen display (OSD) generator for generating, for the time shift function, a progress bar showing a progress state of the storage of the transmitted image in the storage unit within a predetermined storage range.

12. The capsule endoscope system according to claim 11, wherein the progress bar comprises:
a cache bar having a length corresponding to the predetermined storage range, the cache bar showing the progress state; and
an indicator for indicating a current storage location on the cache bar.

13. The capsule endoscope system according to claim 12, wherein the progress state is displayed on the cache bar using at least one of shading and coloring.

14. The capsule endoscope system according to claim 9, wherein the display comprises one or more representative image windows, each of the one or more representative image windows displaying a corresponding one of one or more representative images of a specific image.

15. The capsule endoscope system according to claim 14, wherein the display further comprises one or more information windows, each of the one or more information windows displaying a title, time and related information of the representative image displayed in a corresponding one of the one or more representative image windows.
